(19) United States
(12) Patent Application Publication
(10) Pub. No.: US 2023/0093107 A1
(43) Pub. Date: Mar. 23, 2023

(54) PHYSIOLOGICAL INFORMATION PROCESSING APPARATUS AND PHYSIOLOGICAL INFORMATION PROCESSING METHOD

(71) Applicant: NIHON KOHDEN CORPORATION, Tokyo (JP)

(72) Inventor: Kei KIMURA, Tokorozawa-shi (JP)

(21) Appl. No.: 17/931,179

(22) Filed: Sep. 12, 2022

(30) Foreign Application Priority Data
Sep. 22, 2021 (JP) 2021-154160

(51) Int. Cl. A61B 5/366; A61B 5/021; A61B 5/00

(52) U.S. Cl. CPC A61B 5/366; A61B 5/02108; A61B 5/7221

(57) ABSTRACT

The physiological information processing apparatus: acquires electrocardiogram data of a subject; detects a QRS complex from the electrocardiogram data; acquires pulse wave data of the subject; detects a pacing pulse output from an apparatus worn by the subject; determines whether the pacing pulse is detected in a time period in which the QRS complex is not detected when the time period is equal to or longer than a predetermined time width; determines, based on the pulse wave data, whether a pulse wave is present within predetermined time from a detection time point at which die pacing pulse is detected; and determines, according to a determination that the pulse wave is present within the predetermined time, that the QRS complex is actually present in the time period.

### CROSS-REFERENCE TO RELATED APPLICATIONS

This application is based on and claims priority under 35 USC 119 from Japanese Patent Application No. 2021-154160 filed on Sep. 22, 2021, the contents of which are incorporated herein by reference.

### TECHNICAL FIELD

The presently disclosed subject matter relates to a physiological information processing apparatus and a physiological information processing method. The presently disclosed subject matter further relates to a program that causes a computer to execute the physiological information processing method.

### BACKGROUND ART

When an electrocardiogram waveform of a patient with a cardiovascular implantable electronic device (CIED) such as a pacemaker is measured, a pacing pulse output from the electronic device may be erroneously recognized as a QRS complex by a patient monitor. As a result, reliability of a diagnosis result of a cardiac function of the patient based on electrocardiogram data decreases.

On the other hand, in consideration of a fact that the pacing pulse affects the QRS complex, Patent Literature 1 discloses a processing method for preventing a patient monitor from detecting a QRS complex present near the time of a detected pacing pulse.

### CITATION LIST

### Patent Literature

Patent Literature 1: Japanese Patent No. 2635079

When no QRS complex present near the time of pacing pulse is detected, a healthcare worker cannot accurately grasp an actual state of a cardiac function of a patient with a pacemaker or the like. In this regard, for example, even when the cardiac function of the patient is in a normal state, a time interval between adjacent QRS complexes becomes large when none of a plurality of QRS complexes is detected due to the presence of pacing pulses. As a result, it is assumed that an alarm indicating an abnormality of the cardiac function of the patient is erroneously output from the patient monitor. In this way, from the above viewpoint, there is room for consideration of a processing apparatus that can suitably prevent a situation in which the actually present QRS complex is not detected due to the pacing pulse.

### SUMMARY

An object of the presently disclosed subject mailer is to provide a physiological information processing apparatus and a physiological information processing method that can suitably prevent a situation in which an actually present QRS complex is not detected due to a pacing pulse.

A physiological information processing apparatus according to a first aspect of the presently disclosed subject matter includes:

one or more processors; and

one or more memories configured to store a computer readable command.

In a case where the computer readable command is executed by the processor, the physiological information processing apparatus:
acquires electrocardiogram data of a subject;
detects a QRS complex from the electrocardiogram data;
acquires pulse w ave data of the subject;
detects a pacing pulse output from an apparatus worn by the subject;
determines whether the pacing pulse is detected in a time period in which the QRS complex is not detected when the time period is equal to or longer than a predetermined time width;
determines, based on the pulse wave data, whether a pulse wave is present within predetermined time from a detection time point at which the pacing pulse is detected; and
determines, according to a determination that the pulse wave is present within the predetermined time, that the QRS complex is actually present in the time period.

According to the above configuration, the physiological information processing apparatus determines whether the pacing pulse is present in the time period in which the QRS complex is not detected when the time period is equal to or longer than the predetermined time width. Further, the physiological information processing apparatus determines, according to the determination that the pulse wave related to the QRS complex is present within the predetermined time mm the detection time point at which the pacing pulse is detected, that the QRS complex is actually present in the time period. In this way, it is possible to provide a physiological information processing apparatus that can suitably present a situation in which an actually present QRS complex is not detected doe to a pacing pulse.

A physiological information processing method executed by a computer according to a second aspect of the presently disclosed subject matter includes:

acquiring electrocardiogram data of a subject;

detecting a QRS complex from the electrocardiogram data;

acquiring pulse wave data of the subject;

detecting a pacing pulse output from an apparatus implanted in a body of the subject;

determining whether the pacing pulse is detected in a time period in which the QRS complex is not detected when the time period is equal to or longer than a predetermined time width;

determining, based on the pulse wave data, whether a pulse wave is present within predetermined time from a detection time point at which the pacing pulse is detected; and

determining, according to a determination that the pulse wave is present within the predetermined time, that the QRS complex is actually present in the time period.

There is provided a program that causes a computer to execute the physiological information processing method.

According to the presently disclosed subject matter, it is possible to provide a physiological information processing apparatus and a physiological information processing method that can suitably prevent a situation in which an actually present QRS complex is not detected due to a pacing pulse.

### BRIEF DESCRIPTION OF DRAWINGS

FIG. 1 is a hardware configuration diagram illustrating a physiological information processing apparatus according to the presently disclosed subject matter (hereinafter, simply referred to as the present embodiment).

FIG. 2 is a diagram illustrating an example of a configuration of a sensor interface.

FIG. 3 is a flowchart illustrating a process of obtaining a feature related to a QRS complex from electrocardiogram data.

FIG. 4 is a flowchart illustrating a process of obtaining a feature related to a pulse wave from pulse wave data.

FIG. 5 is a flowchart illustrating a physiological information processing method according to the present embodiment.

FIG. 6 is a diagram illustrating a QRS complex not detected due to a pacing pulse and a pulse wave present within predetermined time from a detection time point of the pacing pulse.

### DESCRIPTION OF EMBODIMENTS

Hereinafter, an embodiment will be described with reference to the drawings. For convenience of description, description of elements having the same reference numerals as those already described in the description of the present embodiment will be omitted.

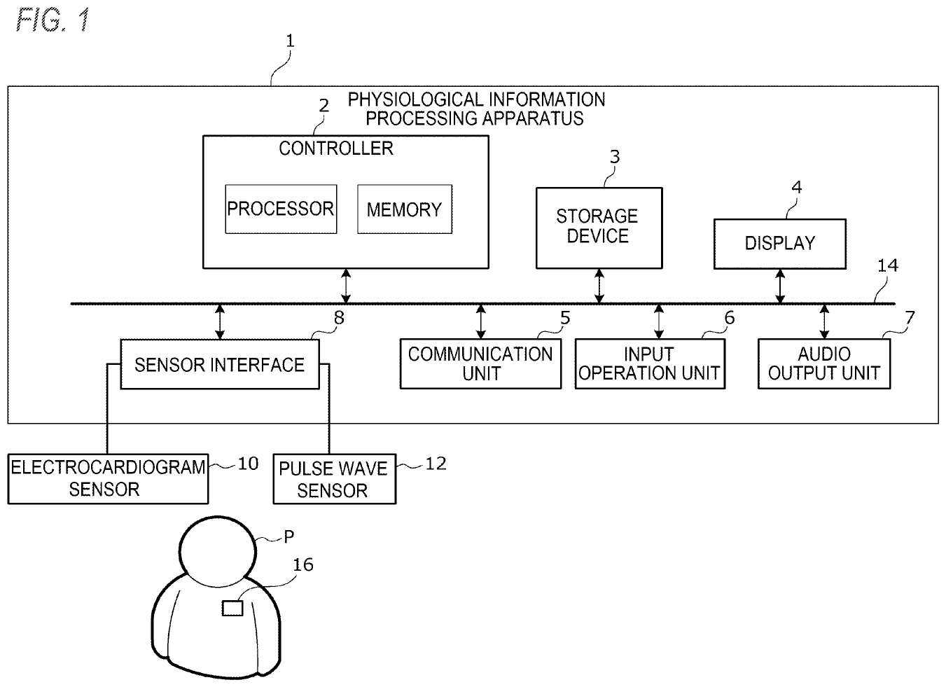

FIG. 1 illustrates a hardware configuration diagram of a physiological information processing apparatus 1 according to the present embodiment. As illustrated in FIG. 1, the physiological information processing apparatus 1 (hereinafter, simply referred to as the processing apparatus 1) includes a controller 2, a storage device 3, a display 4, a communication unit 5, an input operation unit 6, an audio output unit 7, and a sensor interface 8. These components provided in the processing apparatus 1 are communicably connected to each other via a bus 14.

The processing apparatus 1 may be a medical instrument (for example, a patient monitor) that displays physiological information on a subject P, a personal computer, a workstation, a smartphone, a tablet, or a wearable device (for example, an AR glass) worn on a body (for example, an arm, a head) of a healthcare worker.

The subject P wears a pacemaker 16 that is an example of a CIED. The pacemaker 16 is implanted in the body of the subject P, and outputs a pacing pulse that applies electrical stimulation to the heart of the subject P. The timing at which the pacing pulse is output from the pacemaker 16 may overlap the output timing of a QRS complex in an electrocardiogram waveform indicating an electrical activity of the heart of the subject P. The QRS complex on which the pacing pulse is superimposed is generally referred to as a fusion beat or a pseudofusion beat. In this case, the pacing pulse adversely affects the QRS complex, and accordingly reliability of a diagnosis result of a cardiac function of the subject P based on a feature related to the QRS complex may decrease. In this way, the processing apparatus 1 according to the present embodiment does not store data related to the QRS complex present near the pacing pulse on a timeline when the output tuning of the pacing pulse substantially overlaps the output timing of the QRS complex. That is, although the QRS complex present near the pacing pulse on the timeline is actually present cm electrocardiogram dam, the processing apparatus 1 does not detect the QRS complex or store data related to the QRS complex. On the other hand, the processing apparatus 1 according to the present embodiment has a function of determining, based on pulse wave data, whether the QRS complex not detected due to the presence of the pacing pulse is actually present. Details of the determination function will be described later.

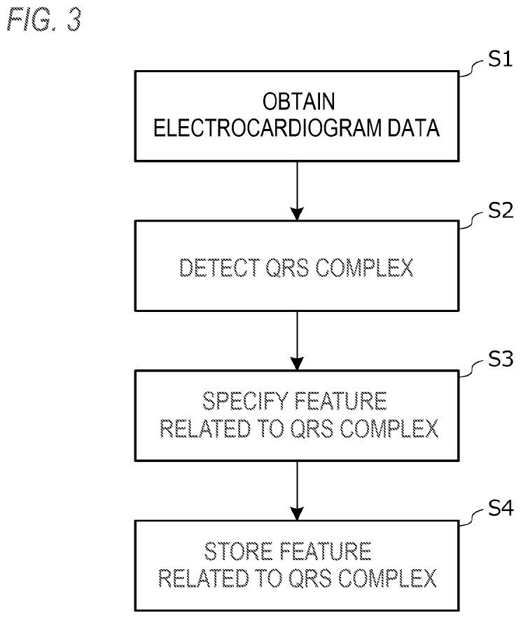

The controller 2 includes one or more memories and one or more processors. The memory stores a computer readable command (program). The memory includes, for example, a read only memory (ROM) that stores various programs and the like, and a random access memory (RAM) having a plurality of work areas in which various programs and the like to be executed by the processor are stored. The processor includes, for example, at least one of a central processing unit (CPU), a micro-processing unit (MPU), and a graphics processing unit (GPU). The CPU may include a plurality of CPU cores. The GPU may include a plurality of GPU cores. The processor may load a designated program from various programs provided in the storage device 3 or the ROM onto the RAM and execute various processes in cooperation with the RAM. In particular, when the processor loads a physiological information processing program for executing a series of processes illustrated in FIGS. 3 to 5 onto the RAM and executes the program in cooperation with the RAM, the controller 2 executes the series of processes illustrated in FIGS. 3 to 5. Details of the physiological information processing program will be described later.

The storage device 3 is, for example, a storage device (storage) such as a hard disk drive (HDD), a solid state drive (SSD), or a flash memory, and stores programs and various data. The physiological information processing program may be provided in the storage device 3. Further, physiological information data (electrocardiogram data, pulse wave data, or the like) indicating physiological information on the subject P may be stored in the storage device 3. For example, electrocardiogram data obtained by an electrocardiogram sensor 10 may be stored in the storage device 3 via the sensor interface 8.

The communication unit 5 connects the processing apparatus 1 to an in-hospital network. Specifically, the communication unit 5 may include various wired connection terminals that communicate with a central monitor or a server provided in the in-hospital network. The communication unit 5 may further include a wireless communication module that performs wireless communication with the central monitor or the server. The communication unit 5 may include, for example, a wireless communication module corresponding to a medical telemeter system. The communication unit 5 may include a wireless communication module corresponding to a wireless communication standard such as Wi-Fi (registered trademark) or Bluetooth (registered trademark) and/or a wireless communication module corresponding to a mobile communication system using a SIM. The in-hospital, network may be, for example, a local area network (LAN) or a wide area network (WAN). The processing apparatus 1 may be connected to the Internet via the in-hospital network.

The display 4 displays information (for example, an electrocardiogram waveform or a pulse wave) related to physiological information data of the subject P obtained in real time, and is, for example, a liquid crystal panel or an organic EL panel. Further, the display 4 displays, on a display screen, an alarm, in accordance with a time interval between adjacent QRS complexes on a timeline. The input operation unit 6 is, for example, a touch panel overlapping the display 4, a mouse, and/or a keyboard. The input operation unit 6 receives an input operation by the healthcare worker and generates an operation signal corresponding to the input operation by the healthcare worker. After the operation signal generated by the input operation unit 6 is transmitted to the controller 2 via the bus 14, the controller 2 executes a predetermined operation in accordance with the operation signal. The audio output unit 7 includes one or more speakers, and outputs an audio alarm in accordance with the time interval between adjacent QRS complexes on the timeline.

The sensor interlace 8 is an interface that connects a vital sensor such as the electrocardiogram sensor 10 and a pulse wave sensor 12 to the processing apparatus 1. The sensor interface 8 may include an input terminal to which a physiological signal output from a vital sensor is input. The input terminal may be physically connected to a connector of the vital sensor. The electrocardiogram sensor 10 obtains an electrocardiogram signal indicating an electrical activity of the heart of the subject P, and includes a plurality of ECG electrodes attached to the subject P. The processing apparatus 1 generates electrocardiogram data based on an electrocardiogram signal output from the electrocardiogram sensor 10 and detects a pacing pulse. The pulse wave sensor 12 obtains a pulse wave signal indicating a pulse wave of the subject P. The pulse wave sensor 12 may include, for example, a light emitter (for example, an LED) that emits red light and/or infrared light and a light detecting element (for example, a photodiode) that detects light that is emitted from the light emitter and that passes through the finger of the subject P. The processing apparatus 1 generates pulse wave data based on a pulse wave signal output from the pulse wave sensor 12. Further, the processing apparatus 1 may obtain information on arterial transcutaneous oxygen saturation (SpO2) and/or blood pressure of the subject P based on the generated pulse wave data.

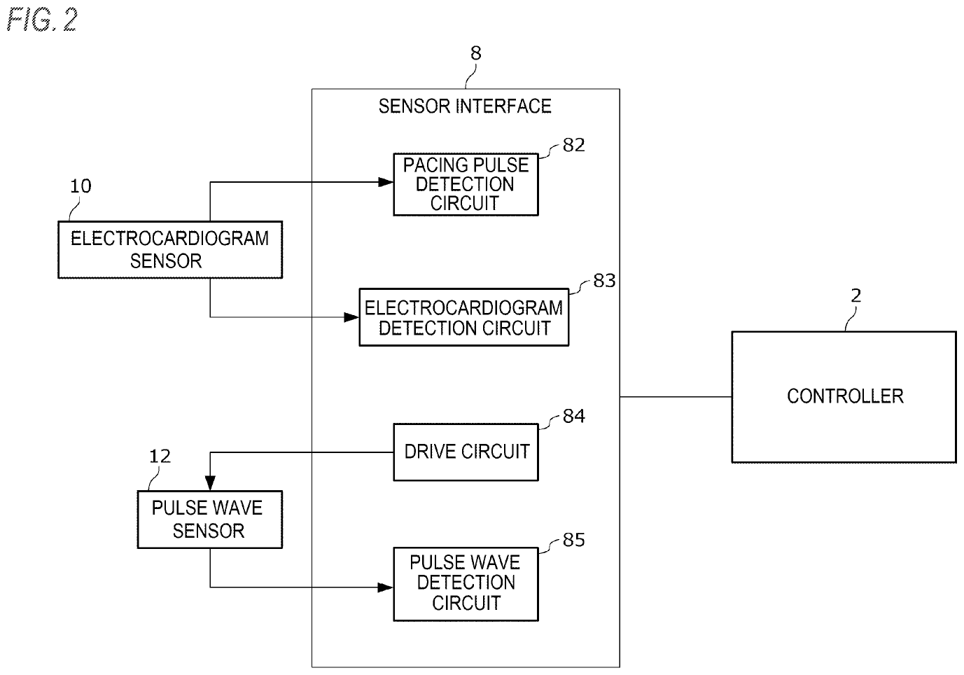

Next, the sensor interface 8 will be described in detail with reference to FIG. 2. As illustrated in FIG. 2, the sensor interlace 8 includes a pacing pulse detection circuit 82, an electrocardiogram detection circuit 83, a drive circuit 84, and a pulse wave detection circuit 85.

The pacing pulse detection circuit 82 detects a pacing pulse based on a high-frequency component (frequency baud of several kHz) of the electrocardiogram signal output from the electrocardiogram sensor 10 The pacing pulse detection circuit 82 includes, for example, at least a high-pass filter and a comparator. The electrocardiogram detection circuit 83 generates electrocardiogram data (digital data) based on a low-frequency component (frequency band of several Hz) of the electrocardiogram signal output from the electrocardiogram sensor 10. The electrocardiogram detection circuit 83 includes at least a differential amplifier circuit, a low-pass filter, and an AD converter. The drive circuit 84 generates a drive signal for driving the pulse wave sensor 12 and then supplies the drive signal to the pulse wave sensor 12. The pulse wave detection circuit 85 generates pulse wave data (digital data) based on the pulse wave signal output from the pulse wave sensor 12. The pulse wave detection circuit 85 includes at least an amplifier circuit, a filter circuit, and an AD converter.

In this way, the sensor interface 8 generates the electrocardiogram data (digital data) based on the electrocardiogram signal (analog signal) output from the electrocardiogram sensor 10, and then transmits the electrocardiogram data to the controller 2. The sensor interface 8 generates the pulse wave data (digital data) based on the pulse wave signal (analog signal) output from the pulse wave sensor 12, and then transmits the pulse wave data to the controller 2.

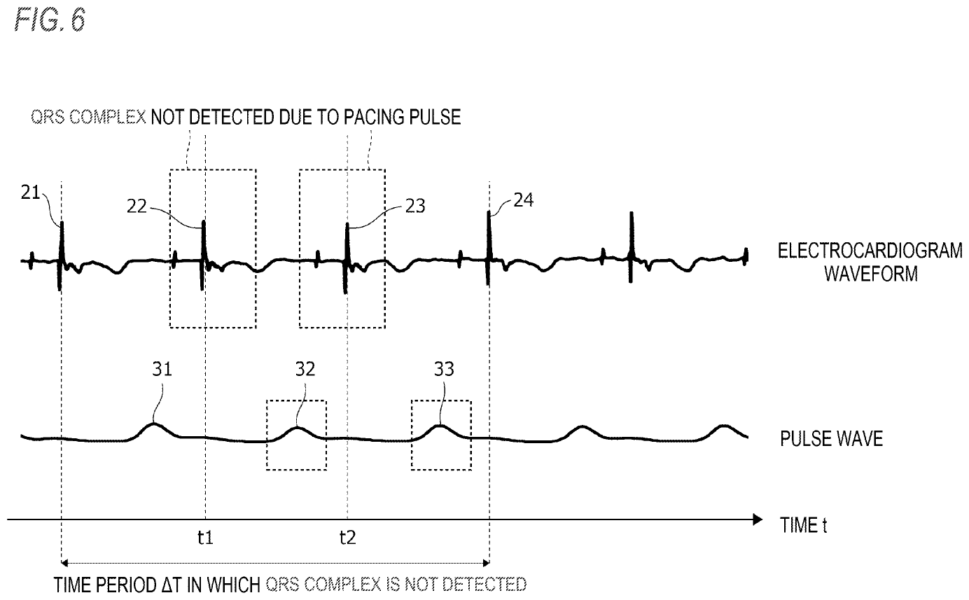

Next, a process of obtaining a feature related to the QRS complex based on electrocardiogram data will be described below with reference to FIG. 3. FIG. 3 is a flowchart illustrating a process of obtaining a feature related to the QRS complex based on electrocardiogram data. As illustrated in FIG. 3, in step S1, the controller 2 obtains electrocardiogram data from the electrocardiogram sensor 10 via the sensor interface 8. In step S2, the controller 2 detects a QRS complex indicating excitation of the ventricle from the electrocardiogram data based on a predetermined detection algorithm. For example, an example of a plurality of QRS complexes is illustrated in an upper part of FIG. 6.

Next, the controller 2 specifies a feature related to the QRS complex for each of the plurality of QRS complexes (step S3). The feature related to the QRS complex includes, for example, at least one of an interval between adjacent QRS complexes on a timeline (specifically, an RR interval indicating an interval between adjacent R waves), a time width of the QRS complex, an amplitude of the QRS complex, a maximum value/minimum value of the QRS complex, and an area of the QRS complex. Thereafter, the controller 2 stores the feature related to the QRS complex in the memory or the storage device 3 (step S4). In this case, the controller 2 may store a moving average value (for example, a moving average value of 16 beats) of the feature related to the QRS complex.

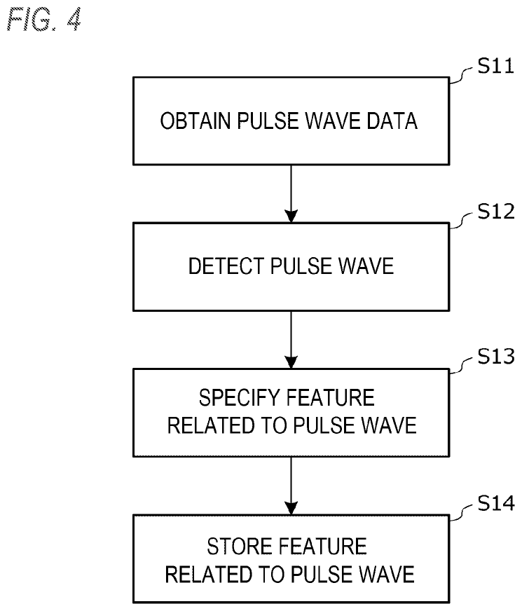

Further, a process of obtaining a feature related to a pulse wave based on pulse wave data will be described below with reference to FIG. 4. FIG. 4 is a flowchart illustrating a process of obtaining a feature related to a pulse wave based on pulse wave data. As illustrated in FIG. 4, in step S11, the controller 2 obtains pulse wave data from the pulse wave sensor 12 via the sensor interface 8. In step S12, the controller 2 detects a pulse wave from the pulse wave data based on a predetermined detection algorithm. For example, an example of a plurality of pulse waves is illustrated in a lower part of FIG. 6.

Next, the controller 2 specifies a feature related to the pulse wave tor each of the plurality of pulse waves (step S13). The feature related to the pulse wave includes, for example, at least one of an amplitude of the pulse wave, a time width of the pulse wave, a maximum value-minimum value of the pulse wave, contraction time of the pulse wave, and delay time of the pulse wave. The “contraction time of the pulse wave” is time from a time point corresponding to a rising point of the pulse wave to a time point corresponding to a peak point of the pulse wave. The “delay time of the pulse wave” corresponds to a time interval between a detection time point at which the pulse wave is detected and a detection time point at which the QRS complex associated with the pulse wave (particularly, the QRS complex appearing immediately before the pulse wave) is detected. More specifically, the “delay time of the pulse wave” may be a time interval between the time point corresponding to the rising point of the pulse wave and a time point corresponding to a rising point of the QRS complex appearing immediately before the pulse wave, or a time interval between the time point corresponding to the peak point of the pulse wave and a time point corresponding to the peak point of the QRS complex appearing immediately before the pulse wave. Thereafter, the controller 2 stores the feature related to the pulse wave in the memory or the storage device 3 (step S14). In this case, the controller 2 may store a moving average value (for example, a moving average value of 16 beats) of the feature related to the pulse wave.

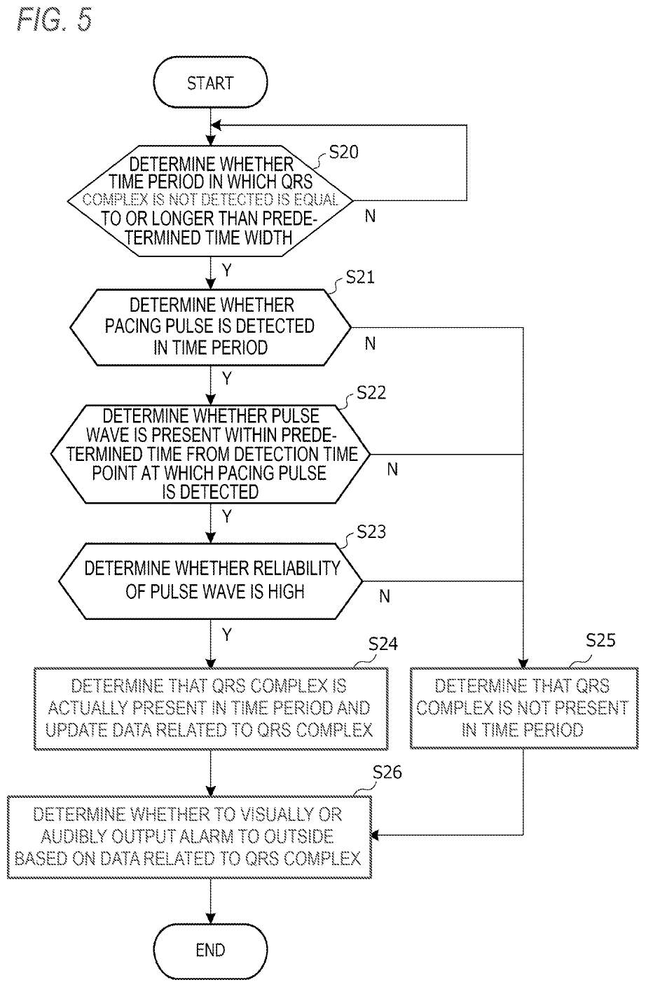

Next, a physiological information processing method according to the present embodiment will be described below with reference to FIGS. 5 and 6. FIG. 5 is a flowchart illustrating the physiological information processing method according to the present embodiment. FIG. 6 is a diagram illustrating QRS complexes 22, 23 not detected due to the pacing pulse and pulse waves 32, 33 present within predetermined lime from detection time points t1, t2 of the pacing pulse. As a precondition, the processing apparatus 1 according to the present embodiment does not detect the QRS complex present near the pacing pulse on a timeline. It is further assumed that the processing apparatus 1 executes an example of the process illustrated in FIG. 5 while executing the series of processes illustrated in FIG. 3 (that is, while obtaining the QRS complex from the electrocardiogram data).

As illustrated in FIG. 5, in step S20, the controller 2 determines whether a time period ΔT in which the QRS complex is not detected is equal to or longer than a predetermined time width. Here, the predetermined time width is, for example, a predetermined value between 200 milliseconds and 2 seconds. When the controller 2 detects that the time period ΔT is equal to or longer than the predetermined time width (Yes in step S20), the controller 2 executes the processing of step S21. On the other hand, when the determination processing of step S20 is No, the controller 2 waits until the time period ΔT is equal to or longer than the predetermined time width.

In the example illustrated in FIG. 6, since the QRS complexes 22, 23 overlap the pacing pulse on the timeline, the QRS complexes 22, 23 are not detected by the processing apparatus 1. That is, the time period ΔT in which the QRS complex is not detected is a time period from a time point corresponding to a peak point of a QRS complex 21 to a time point corresponding to a peak point of a QRS complex 24. In addition, it is assumed that the pacing pulse is detected at the time points t1, t2.

Next, in step S21, the controller 2 determines whether a pacing pulse is detected in the time period ΔT. In particular, the controller 2 determines whether a pacing pulse is detected in the time period ΔT based on information output from the pacing pulse detection circuit 82. When a determination result of step S21 is Yes, the process proceeds to step S22. On the other hand, when the determination result of step S21 is No, the process proceeds to step S25. In this case, the controller 2 determines that the QRS complex is not present in the time period ΔT (step S25), and then executes the processing of step S26.

In step S22, the controller 2 determines, based on pulse wave data, whether a pulse wave is present within predetermined time from a detection time point at which a pacing pulse is detected. Here, the predetermined time is, for example, 1 second. In the example illustrated in FIG. 6, the controller 2 determines whether the pulse wave 32 is present within predetermined time from the detection time point t1, and determines whether the pulse wave 33 is present within predetermined time from the detection time point t2, based on the pulse wave data. When a determination result of step S22 is Yes, the process proceeds to step S23, and when the determination result of step S22 is No, the process proceeds to step S25.

In step S23, the controller 2 determines whether reliability of the pulse wave present within the predetermined time is high. In particular, the controller 2 may determine the reliability of the pulse wave by comparing a feature of the pulse wave present within the predetermined time with a feature of a pulse wave stored in the memory. As described above, the feature of the pulse wave includes at least one of the amplitude of the pulse wave, the time width of the pulse wave, the maximum value minimum value of the pulse wave, the contraction time of the pulse wave, and the delay time of the pulse wave.

For example, the controller 2 may determine that the reliability of the pulse wave 32 is high when a feature of the pulse wave 32 present within the predetermined time satisfies a part or all of the following conditions.

The amplitude of the pulse wave 32 is 0.25 to less than 3 times an average amplitude of the pulse wave stored in the memory.

The delay time of the pulse wave 32 from the detection time point t1 of the pacing pulse to the detection time point of the pulse wave 32 is within ±80 ms of average delay time of the pulse wave stored in the memory.

The delay time of the pulse wave 32 from the detection time point t1 of the pacing pulse to the detection time point of the pulse wave 32 is within ±80 ms of the delay time of the pulse wave 31 adjacent to the pulse wave 32.

The contraction time of the pulse wave 32 is not longer than average contraction time of the pulse wave stored in the memory by 120 ms or more.

The controller 2 may determine the reliability of the pulse wave by determining whether noise is superimposed on the pulse wave present within the predetermined time, in addition to the comparison between the feature of the pulse wave present within the predetermined time and the feature of the pulse wave stored in the memory. For example, when the reliability of the pulse wave 32 is high in a comparison result between the feature of the pulse wave 32 and the feature of the pulse wave stored in the memory and noise is superimposed on the pulse wave, the controller 2 may determine that the reliability of the pulse wave 32 is not high. When the reliability of the pulse wave 32 is high in the comparison result between the feature of the pulse wave 32 and the feature of the pulse wave stored in the memory and noise is not superimposed on the pulse wave 32, the controller 2 may determine that the reliability of the pulse wave 32 is high. In this way, the reliability of the pulse wave is determined in consideration of the noise superimposed on the pulse wave, and thus it is possible to more accurately determine the reliability of the pulse wave.

When a determination result of step S23 is Yes, the process proceeds to step S24, and when the determination result of step S23 is No, the process proceeds to step S25. Next, in step S24, the controller 2 determines that the QRS complex is actually present in the time period ΔT, and updates data related to the QRS complex. In the example illustrated in FIG. 6, the controller 2 can determine that the two QRS complexes 22, 23 are actually present within the time period ΔT after determining that the reliability of the two pulse waves 32, 33 is high.

Further, the controller 2 may update, as the data related to the QRS complex, information related to the time interval between adjacent QRS complexes (in particular, information related to the RR interval). In this regard, the controller 2 may store the detection time point t1 at which the pacing pulse is detected as a detection time point at which the QRS complex 22 is detected (in particular, the time point corresponding to the peak point of the QRS complex 22), and may store the detection time point t2 at which the pacing pulse is detected as a detection time point at which the QRS complex 23 is detected (in particular, the time point corresponding to the peak point of the QRS complex 23).

Next, in step S26, the controller 2 determines whether to visually or audibly output an alarm indicating an abnormality of the cardiac function of the subject P to the outside based on the data related to the QRS complex. In particular, the controller 2 may determine whether to output an alarm to the outside in accordance with a comparison between the time interval between adjacent QRS complexes and a predetermined threshold value Tth. Here, the predetermined threshold value Tth may be set within a range of 3 seconds to 5 seconds, or be a value N times (N>1) the average value of the measured RR interval. For example, when the time interval (RR interval) between adjacent QRS complexes is equal to or greater than the predetermined threshold value Tth, the controller 2 may determine to output an alarm to the outside and then output the alarm to the outside through the audio output unit 7 and/or the display 4. On the other hand, when the time interval between the adjacent QRS complexes is smaller than the predetermined threshold value Tth, the controller 2 determines not to output the alarm to the outside.

The controller 2 may output a plurality of types of alarms to the outside according to the state of the cardiac function of the subject P (severity level of the cardiac function). In this ease, the controller 2 may change the type of an alarm to be output to the outside according to the time interval between adjacent QRS complexes. For example, the controller 2 may output a first alarm to the outside when the time interval is between a first threshold value Tth1 and a second threshold value Tth2. When the time interval is larger than the second threshold value Tth2, the controller 2 may output a second alarm different from the first alarm to the outside.

In the example illustrated in FIG. 6, since it is determined that the QRS complexes 22, 23 not detected clue to the pacing pulse are actually present, the information on the time interval between adjacent QRS complexes is updated. As a result, since the time interval between adjacent QRS complexes does not exceed the predetermined threshold value Tth, no alarm is output from the processing apparatus 1. In this way, when the cardiac function of the subject P is normal, it is possible to suitably prevent a situation in which an alarm indicating an abnormality of the cardiac function of the subject P is erroneously output from the processing apparatus 1. On the other hand, in a processing apparatus in the related art, since it is not determined whether the QRS complexes 22, 23 not detected by the pacing pulse are actually present, the time interval between adjacent QRS complexes exceeds the predetermined threshold value Tth. In this example, since the time interval ΔT between the time point corresponding to the peak point of the QRS complex 21 and the time point corresponding to the peak point of the QRS complex 24 exceeds the predetermined threshold value Tth, an alarm is output from the processing apparatus. As a result, although the cardiac function of the subject P is normal, an alarm indicating an abnormality of the cardiac function of the subject P is erroneously output from the processing apparatus.

As described above, according to the present embodiment, it is possible to suitably prevent a situation in which the actually present QRS complex is not detected due to the pacing pulse. Furthermore, when the cardiac function of the subject P wearing the pacemaker 16 is normal, it is possible to suitably prevent a situation in which an alarm indicating an abnormality of the cardiac function of the subject P is erroneously output. Therefore, it is possible to provide the processing apparatus 1 that can more accurately determine the state of the cardiac function of the subject P wearing the pacemaker.

According to the present embodiment, it is determined that tire QRS complex is actually present in the time period ΔT according to the determination of a high reliability of the pulse wave present within predetermined time (for example, within 1 second) from the detection time point of the pacing pulse. In this way, it is possible to more reliably determine that the QRS complex is actually present within the time period ΔT, and it is possible to more accurately determine the state of the cardiac function of the subject P.

In the description of the present embodiment, the determination processing (step S23) related to the reliability of the pulse wave is executed, but the determination processing of step S23 is not necessarily executed. In this case, after determining in step S22 that the pulse wave is present within predetermined time from the detection time point at which the pacing pulse is detected, the controller 2 may determine that the QRS complex is actually present in the time period ΔT and update the data related to the QRS complex.

In order to implement the processing apparatus 1 according to the present embodiment by software, a physiological information processing program may be pre-loaded into the storage device 3 or the ROM. Alternatively, the physiological information processing program may be stored in a computer-readable storage medium such as a magnetic disk (for example, a HDD or a floppy disk), an optical disk (for example, a CD-ROM, a DVD-ROM, or a Blu-ray (registered trademark) disk), a magneto-optical disk (for example, a MO), or a flash memory (for example, a SD card, a USB memory, or a SSD). In this case, the physiological information processing program stored in the storage medium may be loaded into the storage device 3. Further, the program assembled in the storage device 3 may be loaded onto the RAM, and then the processor may execute the program loaded onto the RAM. In this way, the physiological information processing method according to the present embodiment is executed by the processing apparatus 1.

The physiological information processing program may be downloaded from a computer on a communication network via the communication unit 5. In this case, same or similarly, the downloaded program may be loaded into the storage device 3.

Although the embodiment of the presently disclosed subject mutter is described above, the technical scope of the presently disclosed subject matter should not be construed as being limited to the description of the embodiment. It is understood by those skilled in the art that the present embodiment is an example and various modifications can be made within the scope of the inventions described in the claims. The technical scope of the presently disclosed subject matter should be determined based on the scope of the inventions described in the claims and the scope of equivalents thereof.

1. A physiological information processing apparatus comprising:
one or more processors; and
one or more memories configured to store a computer readable command,
wherein in a case where the computer readable command is executed by the processor, the physiological information processing apparatus is configured to:
acquire electrocardiogram data of a subject;
detect a QRS complex from the electrocardiogram data;
acquire pulse wave data of the subject;
detect a pacing pulse output from an apparatus worn by the subject;
determine whether the pacing pulse Is detected in a time period in which the QRS complex is not detected when the time period is equal to or longer than a predetermined time width;
determine, based on the pulse wave data, whether a pulse wave is present within predetermined time from a detection time point at which the pacing pulse is detected; and
determine, according to a determination that the pulse wave is present within the predetermined time, that the QRS complex is actually present in the time period.

2. The physiological information processing apparatus according to claim 1,
wherein the physiological information processing apparatus is configured to:
update data related to the QRS complex according to the determination that the pulse wave is present within the predetermined time; and
determine whether to audibly or visually output an alarm indicating an abnormality of a cardiac function of the subject to outside based on the data related to the QRS complex.

3. The physiological information processing apparatus according to claim 2,
wherein the data related to the QRS complex includes in formation related to a time interval between adjacent QRS complexes on a timeline, and
wherein the physiological information processing apparatus is configured to determine whether to audibly or visually output the alarm to the outside in accordance with a comparison between the time interval and a predetermined threshold value.

4. The physiological information processing apparatus according to claim 1,
wherein the physiological information processing apparatus is configured to:
determine reliability of the pulse wave when the pulse wave is determined to be present within the predetermined time; and
determine that the QRS complex is actually present in the time period according to a determination that the reliability of the pulse wave is high.

5. The physiological information processing apparatus according to claim 4,
wherein the physiological information processing apparatus is configured to determine the reliability of the pulse wave by comparing a feature of the pulse wave present within the predetermined time with a feature of a pulse wave stored in the memory.

6. The physiological information processing apparatus according to claim 5,
wherein the physiological information processing apparatus is configured to determine the reliability of the pulse wave by comparing the feature of the pulse wave present within the predetermined time with the feature of the pulse wave stored in the memory, and by determining whether noise is superimposed on the pulse wave present within the predetermined time.

7. The physiological information processing apparatus according to claim 5, wherein the feature of the pulse wave includes at least one of:
a feature related to an amplitude of the pulse wave,
a feature related to contraction time of the pulse wave; and
a feature related to delay time of the pulse wave corresponding to a time interval between a detection tune point at which the pulse wave is detected and a detection time point at which the QRS complex associated with the pulse wave or the pacing pulse is detected.

8. The physiological information processing apparatus according to claim 2,
wherein the physiological information processing apparatus is configured to update, according to the determination that the pulse wave is present within the predetermined time, the data related to the QRS complex by storing a detection time point at which the pacing pulse is detected as a detection time point at which the QRS complex is detected.

9. A physiological information processing method executed by a computer, the method comprising:
acquiring electrocardiogram data of a subject;
detecting a QRS complex from the electrocardiogram data;
acquiring pulse wave data of the subject;
detecting a pacing pulse output from an apparatus implanted in a body of the subject;
determining whether the pacing pulse is detected in a time period in which the QRS complex is not detected when the time period is equal to or longer than a predetermined time width;
determining, based on the pulse wave data, whether a pulse wave is present within predetermined time from a detection time point at which the pacing pulse is detected; and
determining, according to a determination that the pulse wave is present within the predetermined time, that the QRS complex is actually present in the time period.

10. The physiological information processing method according to claim 9, further comprising:
updating data related to the QRS complex according to the determination that the pulse wave is present within the predetermined time; and
determining whether to audibly or visually output an alarm indicating an abnormality of a cardiac function of the subject to outside based on the data related to the QRS complex.

11. The physiological information processing method according to claim 10,
wherein the data related to the QRS complex includes information related to a time interval between adjacent QRS complexes on a timeline, and
wherein the method further includes determining whether to audibly or visually output the alarm to the outside in accordance with a comparison between the time interval and a predetermined threshold value.

12. The physiological information processing method according to claim 9, further comprising:
determining reliability of the pulse wave when the pulse wave is determined to be present within the predetermined time, and
wherein the method further includes determining that the QRS complex is actually present in the time period according to a determination that the reliability of the pulse wave is high.

13. A non-transitory computer-readable medium configured to store a program that causes a computer to execute the physiological information processing method according to claim 9.
